(19) United States
(12) Patent Application Publication
(10) Pub. No.: US 2006/0079486 A1
(43) Pub. Date: Apr. 13, 2006

(54) Lipopolymer conjugates

(76) Inventor: Samuel Zalipsky, Redwood City, CA (US)

Correspondence Address: PERKINS COIE LLP, P.O. BOX 2168, MENLO PARK, CA, 94026, US

(21) Appl. No.: 11/245,673

(22) Filed: Oct. 7, 2005

Related U.S. Application Data
(60) Provisional application No. 60/617,585, filed on Oct. 8, 2004.

(51) Int. Cl. A61K 31/685; C07F 9/06; C07J 51/00

(52) U.S. Cl. 514/78; 554/76; 552/506

(57) ABSTRACT

Conjugates of formula I, below, are useful in biomedicinal applications such as delivery of drugs or labeling moieties or as components of liposomes or micelles. In formula I, A is a hydrophilic polymer, each of L and L′ is independently a linker group, B is a lipid moiety; and Z is a diagnostic ligand, a biologically relevant ligand, or a reactive linking moiety, which is generally linked to the phosphorus atom of the conjugate via a nitrogen, oxygen or sulfur atom in Z.

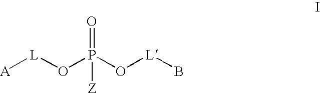

This patent application claims priority to U.S. provisional patent application no. 60/617,585 filed on Oct. 8, 2004, which is incorporated in its entirety herein by reference.

### FIELD OF THE INVENTION

The present invention relates to lipopolymer conjugates in which a hydrophilic polymer is conjugated to a lipid moiety via a phosphoramidate, phosphotriester or phosphothioester group, to which is further conjugated a ligand or a reactive moiety for conjugation of such a ligand. The ligand is a therapeutic or diagnostically relevant molecule.

### REFERENCES

Ghosh, S. S. et al., Bioconjugate Chem. 1:71-76 (1990).
Gryaznov, S. M. and Letsinger, R. L., “Synthesis and properties of oligonucleotides containing aminodeoxythymidine units”, Nucleic Acid Res. 20:3403-9 (1992).
Kirpotin, D., Hong, K., Mullah, N., Papahadjopoulos, D., and Zalipsky, S., “Liposomes with detachable polymer coating: destabilization and fusion of dioleoyl phosphatidylethanolamine vesicles triggered by cleavage of surface-grafted poly(ethylene glycol)”, FEBS Lett. 388(2-3): 115-8 (1996).
Lindh, I. and Stawinski, J., “A general method for the synthesis of glycerophospholipids and their analogs via H-phosphonate intermediates”, J. Org. Chem. 54: 1338-42 (1989).
Lukyanov, A. N. et al., “Micelles from lipid derivatives of water-soluble polymers as delivery systems for poorly soluble drugs”, Adv. Drug Deliv. Rev. 56:1273-89 (2004).
Mlotkowska, B. and Markowska, A., “A new synthesis of thiophosphatidylcholine with carbon-sulfur-phosphorus bond”, Liebigs Annalen der Chemie 2:191-3 (1988).
Solodin, I. et al., “Synthesis of phosphotriester cationic phospholipids”, Synlett 457-8 (1996).
Woodle, M. C., “Poly(ethylene glycol)-grafted liposome therapeutics”, in POLY(ETHYLENE GLYCOL) CHEMISTRY AND BIOLOGICAL APPLICATIONS, J. M. Harris and S. Zalipsky, Eds., ACS Symp. Series 680, pp. 60-81, American Chemical Soc., Washington, D.C. (1997).
Zalipsky, S., “Synthesis of an end-group functionalized polyethylene glycol-lipid conjugate for preparation of polymer-grafted liposomes”, Bioconjug Chem. 4(4):296-9 (1993).
Zalipsky, S., Mullah, N., Harding, J. A., Gittelman, J., Guo, L., and DeFrees, S. A., “Poly(ethylene glycol)-grafted liposomes with oligopeptide or oligosaccharide ligands appended to the termini of the polymer chains”, Bioconjug Chem. 8(2): 111-8 (1997).
Zalipsky, S., Mullah, N., and Qazen, M., “Preparation of poly(ethylene glycol)-grafted liposomes with ligands at the extremities of polymer chains”, Meth. Enzymol. 387:50-69 (2004).

### BACKGROUND OF THE INVENTION

Lipopolymers, in particular mPEG-PE (polyethylene glycol-phosphatidyl ethanolamine) conjugates, have been used extensively in various liposomal and micellar drug delivery formulations (see e.g. Woodle et al., 1997, and Lukyanov et al., 2004, respectively). Conventionally, PEG-lipid conjugates are prepared by linking a polyethylene glycol, such as mPEG, to the amino group of a diacyl phosphatidyl ethanolamine (PE). Several such mPEG-PE conjugates are commercially available, for example, mPEG_{2K}-DSPE, derived from distearoyl PE, is widely used as the principal excipient in STEALTH® liposome formulations. Ligand-PEG-lipid conjugates in which the ligand is linked to the free terminus of the PEG chain have been developed for use in targeted delivery of liposomes (Zalipsky et al., 2004).

Other conjugates are described, for example, in U.S. Pat. No. 5,359,030 (Ekwuribe, 1994), which is directed primarily to conjugates of peptides with nonionic detergents (PEG-hydrophobe adducts). The disclosed structures of the conjugates include the lipid-hydrophobe-ligand structure described above, in which a lipid is linked to a hydrophilic moiety (the nonionic detergent) which in turn is linked to a ligand (peptide) at its other terminus. Alternatively, a more complex structure is disclosed in which the ligand is linked to a lipid and to two hydrophobic moieties, one of which is further linked to another lipid. The nonionic detergents in these conjugates are synthetic materials which tend to be of heterogeneous composition.

### SUMMARY OF THE INVENTION

In one aspect, the invention provides a compound of formula I: 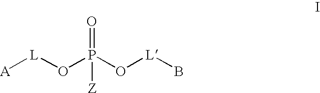
where

A is a hydrophilic polymer,

each of L and L′ is independently a linker group;

B is a lipid moiety; and

Z is selected from the group consisting of a diagnostic ligand, a biologically relevant ligand, and a reactive linking moiety, wherein said reactive linking moiety is not hydroxy (—OH), oxide (—O⁻), or 2-aminoethoxy (—OCH₂CH₂NH₂).

In selected embodiments, the lipid moiety B is selected from a fatty acid, a sterol, a diether lipid, and a diacyl lipid. When B is a diacyl lipid, the compound is preferably of the formula II: 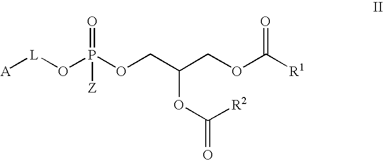
where each of R¹ and R² is independently alkyl or alkenyl having 4-24 carbon atoms. In one embodiment, each of R¹ and R² is C₁₇H₃₅ (a distearoyl lipid).

The ligand or reactive linking moiety Z is preferably linked to the phosphorus atom via a nitrogen, oxygen or sulfur atom in Z. As noted above, the reactive linking moiety is not hydroxy (—OH), oxide (—O⁻), or 2-aminoethoxy (—OCH₂CH₂NH₂); preferably, it is not an aminoalkoxy group (of which 2-aminoethoxy is one example). In preferred embodiments, Z is linked to P via a nitrogen atom in Z, forming a phosphoramidate linkage.

When Z is reactive linking moiety, it may be of the form —NH—(CH₂)ₙ—X, where n is 2 to 6, and X includes a conjugation-prone functional group, such as amino, mercapto, hydroxy, disulfide, aldehyde, ketone, maleimide, hydrazide, other carboxylic acid derivatives, including activated esters, such as succinimidyl (NHS) ester, or a leaving group. Exemplary leaving groups include chloride, bromide, alkylsulfonate, arylsulfonate, and nitrophenylcarbonate. In selected embodiments, X is selected from amino, maleimide, hydrazide, and a succinimidyl (NHS) ester. In one embodiment, n is 3 and X is —NH₂. In another embodiment, n is 3 and X is a succinimidyl ester.

Z may also comprise a diagnostic ligand, such as a fluorescent compound, e.g. fluorescein or coumarin, or a biologically relevant ligand, such as a therapeutic agent (drug) or targeting moiety. Structurally, the ligand can be selected from a polypeptide, a protein, a polynucleotide, and a small molecule compound. In one embodiment, the ligand is a therapeutic polypeptide or protein, in another embodiment, it is a therapeutic small molecule compound.

The hydrophilic polymer A, in one embodiment, is a polyethylene glycol (PEG), preferably having 2 to about 120 repeating ethylene glycol units. The PEG polymer is typically terminated with an alkoxy group, such a methoxy, or a reactive group, such as those described above for X, e.g. hydrazide (H₂N—NH—(CO)—), amino, disulfide, maleimido, nitrophenylcarbonate, or NHS ester.

In selected embodiments, each of the linkages L and L′ is independently an alkyl, aryl, or aralkyl moiety, which may be flanked on one or both sides by a group Y, where Y is (i)-W—(C═O)-Q-, (ii)-W—(C═O)—, (iii)-W—, and (iv) disulfide, where W and Q are independently selected from oxygen, NH, and a direct bond. Preferably, alkyl is lower alkyl, and aryl is a monocyclic or bicyclic, more preferably monocyclic, group.

In another aspect, the invention provides a liposome comprising a compound of formula I or II above, preferably in an amount of 1 to about 50 mole percent of the total lipid content of the liposome.

The invention also provides a method for oral delivery of a therapeutic agent, by administering orally to a subject a conjugate of formula I or II as described above, where Z comprises the therapeutic agent. In this aspect, the relative sizes of the moieties A and B (the hydrophilic polymer and the lipid) can be adjusted to give an HLB (hydrophilic-lipophilic balance) that is favorable to oral delivery. Preferably, Z further comprises a linkage to the phosphorus atom of the conjugate which is cleavable in vivo. In additional preferred embodiments, B is a diacyl lipid, such that the conjugate has the formula II, and A is a polyethylene glycol.

These and other objects and features of the invention will be more fully appreciated when the following detailed description of the invention is read in conjunction with the accompanying drawings.

### BRIEF DESCRIPTION OF THE DRAWINGS

FIG. 1 is a synthetic scheme showing the preparation of a lipopolymer conjugate derived from mPEG-DSPE and containing a detectable ligand, 7-hydroxycoumarin, in accordance with one embodiment of the invention, and

FIG. 2 is a synthetic scheme showing the preparation of a lipopolymer conjugate containing a protein ligand, in accordance with another embodiment of the invention.

### DETAILED DESCRIPTION OF THE INVENTION

I. Definitions

“Alkyl” refers to a monovalent residue containing carbon and hydrogen, which may be linear or branched. Examples of alkyl groups are methyl, ethyl, n-butyl, t-butyl, n-heptyl, and isopropyl. “Cycloalkyl” refers to a fully saturated cyclic monovalent radical containing carbon and hydrogen, preferably having three to seven, more preferably five or six, ring carbon atoms, which may be further substituted with alkyl. Examples of cycloalkyl groups include cyclopropyl, methyl cyclopropyl, cyclobutyl, cyclopentyl, ethylcyclopentyl, and cyclohexyl.

“Lower alkyl” refers to an alkyl radical of one to six carbon atoms, as exemplified by methyl, ethyl, n-butyl, i-butyl, t-butyl, isoamyl, n-pentyl, and isopentyl. In selected embodiments, a “lower alkyl” group has one to four carbon atoms.

An “acyl” group is an organic radical derived from an organic acid by the removal of the carboxylic hydroxyl group. For example, an acyl group derived from a carboxylic acid has the form R—(C═O)—, where R is an alkyl group, which may be a lower alkyl group. Other acyl groups include those of the form R—X(C═O)—, where X is O, S, or NH.

“Hydrocarbyl” encompasses groups consisting of carbon and hydrogen, i.e. alkyl, alkenyl, alkynyl, cycloalkyl, cycloalkenyl, and non-heterocyclic aryl.

“Aryl” refers to a substituted or unsubstituted monovalent aromatic radical having a single ring (e.g., phenyl), two condensed rings (e.g., naphthyl) or three condensed rings (e.g. anthracyl or phenanthryl). Monocyclic groups (also referred to as mononuclear) are generally preferred. The term also includes heteroaryl groups, which are aromatic ring groups having one or more nitrogen, oxygen, or sulfur atoms in the ring, such as furyl, pyrrole, pyridyl, and indole. By “substituted” is meant that one or more ring hydrogens in the aryl group is replaced with a halide such as fluorine, chlorine, or bromine, with a lower alkyl group containing one or two carbon atoms, or with nitro, amino, methylamino, dimethylamino, methoxy, halomethoxy, halomethyl, or haloethyl. Preferred substituents, when present, include fluorine, chlorine, methyl, ethyl, and methoxy.

“PEG” refers to polyethylene glycol, a polymer having the repeating unit (CH₂CH₂O)ₙ, where n is preferably about 10 to about 2300, which corresponds to molecular weights of about 440 Daltons to about 100,000 Daltons. The polymers are typically water soluble over substantially the entire molecular weight range. For conjugation to a polypeptide, a preferred range of PEG molecular weight is from about 2,000 to about 50,000 Daltons, more preferably from about 2,000 to about 40,000 Daltons. The PEG may be end capped with any group that does not interfere with the conjugation reactions described herein, e.g. hydroxyl, ester, amide, thioether, alkoxy, or a variety of reactive groups blocked with appropriate protecting moieties. A common end capped PEG is methoxy PEG (mPEG). While PEG homopolymers are preferred, the term may also include copolymers of PEG with another monomer. This could be, for example, another ether forming monomer, such as propylene glycol.

A “phosphoramidate” linkage refers to a linkage of the form —O—P(═O)(NRR′)—O—, where each of R and R′ represents either hydrogen or a substituent which is linked to N via a carbon atom. A “phosphothioester” linkage, also referred to herein as a “thiophosphate”, refers to a linkage of the form —O—P(═O)(SR)—O—, where R represents a substituent which is linked to S via a carbon atom.

A “conjugation-prone” or “reactive” functional group on a molecule is one that is effective, under conventional conditions of synthetic organic chemistry, to form a covalent linkage with a functional group on another molecule. Such conditions, for example, are those which will not adversely affect non-reacting positions of either molecule.

“Vesicle-forming lipids” refers to amphipathic lipids which have hydrophobic and polar head group moieties, and which can form spontaneously into bilayer vesicles in water, as exemplified by phospholipids, or are stably incorporated into lipid bilayers, with the hydrophobic moiety in contact with the interior, hydrophobic region of the bilayer membrane, and the polar head group moiety oriented toward the exterior, polar surface of the membrane. Such vesicle-forming lipids typically include one or two hydrophobic acyl hydrocarbon chains or a steroid group and may contain a chemically reactive group, such as an amine, acid, ester, aldehyde or alcohol, at the polar head group. Examples include phospholipids, such as phosphatidyl choline (PC), phosphatidyl ethanolamine (PE), phosphatidic acid (PA), phosphatidyl inositol (PI), and sphin-gomyelin (SM), where the two hydrocarbon chains are typically between about 14-22 carbon atoms in length, and have varying degrees of unsaturation. Other vesicle-forming lipids include glycolipids, such as cerebrosides and gangliosides, and sterols, such as cholesterol.

The term “pharmaceutically acceptable salt” encompasses, for example, carboxylate salts having organic or inorganic counterions, such as alkali or alkaline earth metal cations (e.g. lithium, sodium, potassium, magnesium, barium or calcium), ammonium; or organic cations, for example, dibenzylammonium, benzylammonium, 2-hydroxyethylammonium, bis(2-hydroxyethyl) ammonium, phenylethylbenzylammonium, and the like. Other cations include the protonated forms of basic amino acids such as glycine, ornithine, histidine, phenylglycine, lysine, and arginine.

The term also includes salts of basic groups, such as amines, having a counterion derived from an organic or inorganic acid. Such counterions include chloride, sulfate, phosphate, acetate, succinate, citrate, lactate, maleate, fumarate, palmitate, cholate, glutamate, glutarate, tartrate, stearate, salicylate, methanesulfonate, benzenesulfonate, sorbate, picrate, benzoate, cinnamate, and the like.

A “pharmaceutically acceptable carrier” is a carrier suitable for administering the conjugate to a subject, including a human subject, as a pharmaceutical formulation. The carrier is typically an aqueous vehicle, such as aqueous saline, dextrose, glycerol, or ethanol. Inactive ingredients, such as buffers, stabilizers, etc., may be included in the formulation. An “aqueous vehicle” as used herein has water as its primary component but may include solutes as just described. Cosolvents such as alcohols or glycerol may also be present.

Solid formulations, which may also be used, typically include inactive excipients such as mannitol, lactose, starch, magnesium stearate, sodium saccharine, talcum, cellulose or cellulose ethers, glucose, gelatin, sucrose, magnesium carbonate, and the like. The conjugate may also be formulated as a suspension in a lipid or phospholipid, in a liposomal formulation, or in a transdermal or inhalable formulation, according to methods known in the art.

II. Lipopolymer Conjugates

A. Structure and Properties

In one aspect, the invention is directed to lipopolymer conjugates of structure I: 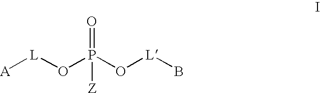
where

A is a hydrophilic polymer;

each of L and L′ is independently a linker;

B is a lipid moiety; and

Z is selected from the group consisting of a therapeutic agent, a diagnostic agent, and a reactive linking moiety.

Not included are compounds in which the reactive linking moiety is hydroxy (—OH), oxide (—O⁻), or 2-aminoethoxy (—OCH₂CH₂NH₂). Preferably, it is not an aminoalkoxy group (of which 2-aminoethoxy is one example).

Exemplary hydrophilic polymers (A) include polyvinylpyrrolidone, polyvinylmethylether, polymethyloxazoline, polyethyloxazoline, polyhydroxypropyloxazoline, polyhydroxypropyl-methacrylamide, polymethacrylamide, polydimethyl-acrylamide, polyhydroxypropylmethacrylate, polyhydroxyethylacrylate, hydroxymethylcellulose, hydroxyethylcellulose, polyethyleneglycol, polyaspartamide, copolymers of the above-recited polymers, and polyethyleneoxide-polypropylene oxide copolymers. Polymers which are fully water soluble at body temperature and fully biocompatible are preferred. Properties and reactions of many of these polymers are described in U.S. Pat. Nos. 5,395,619 and 5,631,018.

In preferred embodiments, the hydrophilic polymer (A) is a poly(alkylene oxide), more preferably a PEG (polyethyleneglycol) polymer, as defined above. Preferably, the PEG polymer has 2 to about 120 repeating ethylene glycol units. Its remote terminus is typically capped with an alkoxy group or a reactive group, e.g. as described for the group X below.

The lipid moiety (B) is a water-insoluble molecule having at least one alkyl or acyl chain containing at least about eight carbon atoms, preferably about 8-24 carbon atoms, or, alternatively, a steroid nucleus. Vesicle-forming lipids are preferred. Exemplary lipids include phospholipids, having a single hydrocarbon chain or, preferably, two hydrocarbon chains, where the hydrocarbon chains are typically between about 4-24, preferably about 8-24, and more preferably about 12-24, carbon atoms in length, and have varying degrees of unsaturation. Other suitable lipids include glycolipids, such as cerebrosides and gangliosides, and steroids, such as cholesterol or cholesterylamine.

In selected embodiments, the lipid moiety B is selected from a fatty acid, a sterol, a diether glyceryl lipid (having two ether-linked hydrocarbon chains), and a diacyl glyceryl lipid (having two acyl-linked hydrocarbon chains). In one embodiment, the lipid moiety B is a diacyl glyceryl lipid, such the that lipopolymer conjugate has the formula II: 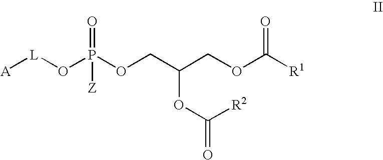
where each of R¹ and R² is independently alkyl or alkenyl having 4 to about 24 carbon atoms, preferably about 6-24 carbon atoms, and more preferably about 12-24 carbon atoms. In one embodiment, each of R¹ and R² is C₁₇H₃₅ (distearoyl).

The group Z attached to the phosphorus atom group may include a therapeutic or diagnostic ligand, e.g. a drug or a targeting, binding or labeling moiety. Typically, Z also includes a short linker group, such as described below for L and L′, connecting the ligand moiety, which may be, for example, a protein, polysaccharide, nucleic acid, oligonucleotide, oligonucleotide analog, or small molecule compound, to the phosphor us atom.

Examples of targeting or binding moieties include biotin, folate, pyridoxal, growth factors, such as vascular endothelial growth factor (VEGF), epidermal growth factor (EGF), and fibroblast growth factor (FGF), cytokines, CD4, and chelators, such as DTPA. Other targeting moieties include those described in U.S. Pat. Nos. 6,660,525 and 6,043,094, which are incorporated herein by reference. Preferred labeling moieties include fluorescent compounds such coumarin and its derivatives, fluorescein and its derivatives, and others known in the art.

Structurally, the ligand may be selected from polymeric or oligomeric biomolecules, e.g. proteins, polysaccharides, nucleic acids, oligonucleotides, oligonucleotide analogs, or small molecule compounds. A “small molecule” compound may be defined broadly as an organic, inorganic, or organometallic compound which is not a polymer or oligomer. Typically, such compounds have molecular weights of less than 1000, or, in one embodiment, less than 500 Da.

Preferably, the group Z is linked to the phosphorus atom via a nitrogen, oxygen or sulfur atom in Z, resulting in a phosphoramidate, phosphotriester, or phosphothioester linkage, respectively. Preferably, the group Z is linked to the phosphorus atom via a nitrogen atom in Z, resulting in a phosphoramidate linkage.

When Z is a reactive linking moiety, it preferably comprises a short chain of atoms (i.e. 1 to about 8 atoms in length, preferably 2 to 6 atoms in length) terminating in a reactive group X, where X is a nucleophilic or electrophilic group effective to react with another group, e.g. on a therapeutic or diagnostic moiety, to form a covalent bond. In one embodiment, Z is a reactive moiety of the form —NH—(CH₂)ₙ—X, where n is 2 to about 6, preferably 2 to 4, and X is a conjugation-prone functional group such as amino, mercapto, disulfide, aldehyde, ketone, maleimide, hydrazide, other carboxylic acid derivatives, etc. The group X may include a leaving group, such as, for example, chloride, bromide, alkylsulfonate, arylsulfonate, succinimidyl ester, or nitrophenylcarbonate. In one embodiment, n is 3 and X is —NH₂, such that Z is 3-aminopropylamine.

The ligand or linking group Z may comprise an in vivo cleavable moiety, such as an ester, carbamate, carbonate, or disulfide, effective to release the ligand from the conjugate in vivo, as discussed further below.

The linkers L and L′ (see structure II) are, in general, storage-stable linkages between the phosphate (or phosphoramidate or phosphothioester, as the case may be) oxygen atoms and the hydrophilic polymer and lipid group, respectively. Preferably, each of L and L′ is independently an alkyl, aryl, or aralkyl moiety, which may be flanked on one or both sides by a group Y, where Y is (i)-W—(C═O)-Q-, (ii)-W—(C═O)—, (iii)-W—, and (iv) disulfide, where W and Q are independently selected from oxygen, NH, and a direct bond. Accordingly, various embodiments of L and/or L′ include a direct bond, an alkyl group, an ether, an ester, an amide, a carbamate, a carbonate, a disulfide, and combinations of any of these with an alkyl or aryl group. The alkyl group is preferably lower alkyl, and the aryl group is preferably mononuclear or binuclear, more preferably mononuclear. Preferred linkers include an ether, an ester, an amide, a carbamate, a carbonate, or a disulfide, in combination with an alkyl group 2 to 4 atoms in length. Also preferred is a dithiobenzyl linker, as described, for example, in U.S. Pat. No. 6,342,244.

In selected embodiments, at least one of L and L′ is cleavable in vivo. Such cleavable linkages include esters and carbonates, which are enzymatically or hydrolytically cleavable, and disulfides, which can be cleaved in vivo by reductive species such as cysteine or glutathione.

The lipopolymer conjugates as described herein have various properties which make them useful as delivery vehicles for the attached ligands. Because the ligand (e.g., a therapeutic agent) is attached near the junction of the lipid and hydrophilic polymer chains in these conjugates, the ligand is likely to be more shielded by the hydrophilic polymer (e.g., PEG) than in prior art conjugates, in which the ligand is normally attached at the terminus of the PEG chain. Benefits of such shielding include longer circulation time and reduced degradation of the ligand.

The positioning of the ligand near the lipid head group also provides useful reagents for studying liposomal lipid insertion, when the ligand is a detectable group.

Furthermore, the hydrophilic-lipophilic balance (HLB) of the lipopolymer conjugates can be adjusted by varying the fatty acid (lipid) and/or the hydrophilic polymer (e.g. PEG) chain lengths. For example, the HLB can be modified for improved membrane penetration, which is beneficial for oral and CNS delivery of attached drugs.

Accordingly, the invention also provides a method of tailoring the hydrophilic-lipophilic balance of a carrier for a drug, by providing a carrier of formula I or II above, where Z is the drug, and the relative sizes of A and B are effective to give a desired HLB for the carrier. In the carrier, A is preferably a PEG polymer.

B. Preparation of the Lipopolymer Conjugates

The conjugates of the inventions may be prepared from an intermediate phosphodiester or phosphotriester lipopolymer structure of the general form A-L-O—P(═O)OR—O-L′—B, where A, B, L, and L′ are as defined above, and OR is oxide, hydroxy, or lower alkoxy, such as methoxy.

When the lipid B is a diacyl glyceryl lipid, such as in structure II above, such an intermediate can be prepared from a diacyl glyceryl phospholipid, many of which are naturally occurring, commercially available, and/or readily prepared by known methods. Various methods have been described in the art for attaching hydrophilic polymers, particularly PEG polymers, to phospholipids. See, for example, Zalipsky, 1993, Kirpotin et al., 1996, Zalipsky et al., 1997. Such PEG-phospholipid compounds may also be commercially available, for example, various PEGylated phosphatidyl ethanolamines, such as mPEG-DSPE (distearoyl phosphatidylethanolamine), are available from Avanti Polar Lipids (Alabaster, Ala.).

When B is not derived from a phospholipid, the phosphodiester (or phosphotriester when OR is alkoxy) A-L-O—P(═O)OR—O-L′-B can be prepared by conventional methods, which may employ, for example, a phosphoramidite intermediate, as commonly employed in oligonucleotide synthesis. Accordingly, in one embodiment of this procedure, a moiety B-L′—OH (lipid moiety with hydroxyl functionality) is reacted with a reagent P(NR₂)(OMe)Cl, where R is typically isopropyl, to form the phosphoramidite P(NR₂)(OMe)—O-L′—B. This intermediate is then reacted with a moiety A-L-OH (hydrophilic polymer with hydroxyl functionality) to form the phosphite triester A-LO-P(OMe)—O-L′—B, which can be oxidized to the phosphate triester. Treatment with base or acid gives the phosphate or phosphoric acid, respectively, if desired.

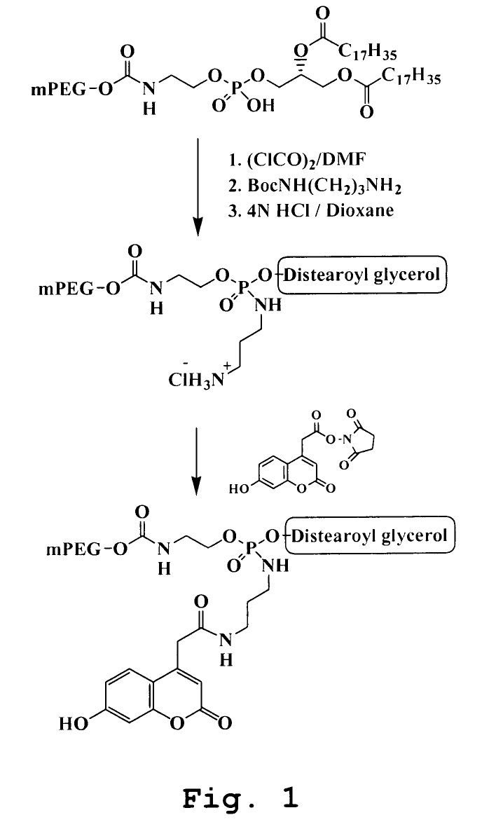

In one embodiment, the group Z is attached to the phosphorus atom in the conjugate via a nitrogen atom in Z, forming a phosphoramidate linkage. Example 1, below, describes linking of a diamine to mPEG-DSPE via formation of a phosphoramidate linkage between one amine terminus and the phosphate head group, leaving the other amine terminus available for further derivatization or conjugation (see FIG. 1). In this procedure, the starting phosphodiester is activated with oxalyl chloride to produce a phosphoryl chloride intermediate, which is reacted without isolation with the amine reagent (in this example, 1-Boc-protected 1,3-diaminopropane). The phosphodiester may also be activated for reaction with an amine by other reagents, such as a carbodiimide in the presence of imidazole (Ghosh et al., 1990). The amino-derivatized lipopolymer conjugate is produced by removal of the Boc protecting group.

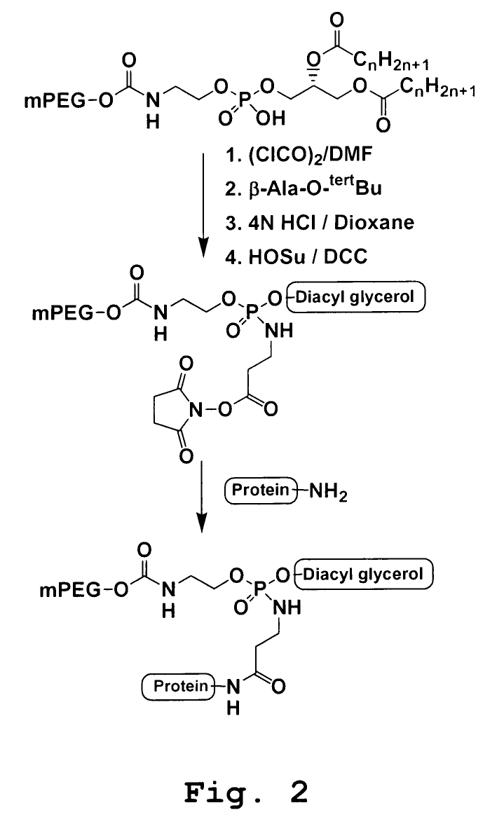

Similar procedures can be used to link other primary or secondary amines to a phosphodiester lipid, forming stable phosphoramide diester conjugates. The linker group Z may have various functionality at the free terminus. For example, an amino acid ester, e.g. β-alanine tert-butyl ester, can be used to provide a free carboxylic acid after acidolytic deprotection, as described in Example 2 and depicted in FIG. 2. The terminal functional group can then be utilized for attachment of a variety of ligands, as described above, e.g. peptides, proteins, polynucleotides, saccharides, targeting groups, chelators, etc., using synthetic methods known in the art.

In the exemplary procedure illustrated in FIG. 1, the amino group of the aminopropane phosphoramidate is deprotected and coupled with a succinimidyl ester of 7-hydroxycoumarin-4-acetic acid, resulting in a fluorescently labeled lipopolymer (designated mPEG-7HC-DSPam). In the procedure illustrated in FIG. 2, a terminal carboxylate, attached via reaction with β-alanine, as described above, is activated as an NHS ester, following by conjugation to an amino group of a protein.

Phosphotriester-linked lipopolymers of the invention can be obtained, for example, by condensing a phosphodiester, such as mPEG-DSPE, with R—OH. The reaction can be mediated by methanesulfonyl chloride or toluenesulfonyl chloride in 2,6-lutidine, e.g. as described by Solodin et al. (1996). The R residue of R—OH can contain a masking group that can be further derivatized.

Mlotkowska & Markowska (1998) report that reaction of a phosphite triester having a methoxy group (i.e., RO—P(OMe)—OR′) with an N-thiolated succinimide (i.e. where the ring nitrogen is substituted with —SR″) produces a thiophosphate, RO—PO(SR″)—OR′.

The conjugates of the invention can also be readily obtained via an H-phosphonate diester intermediate, of the general form A-L-O—P(═O)H—O-L′-B, where A, B, L, and L′ are as defined above. Such an intermediate can be prepared using methods described by Lindh and Stawinski (1989). For example, treatment of diacyl glycerol with phosphorus trichloride/imidazole followed by aqueous workup produces 1,2-diacyl-sn-glycero-3-H-phosphonate in high yield. This H-phosphonate can be linked to a hydrophilic polymer such as mPEG-OH, using pivaloyl chloride, resulting in an H-phosphonate diester-linked mPEG-lipid.

The H-phosphonate group can be readily converted to a phosphoramidate by coupling with an appropriate amine in CCl₄/triethylamine (see, for example, Gryaznov and Letsinger, 1992). This reaction can be used to link any amino-containing reporter or drug moiety.

The H-phosphonate group is also readily convertible into a thiophosphate by a simple treatment with sulfur (Lindh & Stawinski, 1998). The thiophosphate can be further functionalized and labeled with various reporter groups by direct S-alkylation.

As noted above, the lipid-hydrophilic polymer portion of the molecule may be prepared with one or more in vivo cleavable linkages (typically in the L or L′ moieties), such that either the lipid, the hydrophilic polymer, or both are released from the molecule after a certain amount of time in circulation. See, for example, Kirpotin et al., 1996, which describes in vivo cleavage of PEG from PEG-DSPE in liposomes.

The group Z may be a group effective to provide a further linking moiety, or it may be a diagnostic or therapeutic agent. In one embodiment, the linking group Z contains an in vivo cleavable linkage, such that the attached agent is released from the lipopolymer portion of the molecule after a certain amount of time in circulation, preferably after delivery to a target site.

C. Cleavable Linkages

As noted above, the lipopolymer-ligand conjugates described herein may be prepared with one or more in vivo cleavable linkages, such that one or more of the lipid, the hydrophilic polymer, and the ligand (e.g. drug) are released from the conjugate after a certain amount of time in circulation. Such cleavable linkages include esters and carbonates, which are enzymatically or hydrolytically cleavable, and disulfides, which can be cleaved in vivo by reductive species such as cysteine or glutathione. Linkages can be designed for more rapid or for delayed cleavage, according to methods known in the art, including choice of linkage, the use of intramolecular cleavage, and/or modification of steric or electronic properties at or near the cleavage site. See, for example, U.S. Pat. No. 6,342,244, incorporated herein by reference, which describes modulation of cleavage rate by steric effects at the cleavage site.

Different portions of the conjugate can prepared with cleavable linkages, according to the desired change in structure of the conjugate in vivo. See, for example, Kirpotin et al., 1996, which describes in vivo cleavage of PEG from PEG-DSPE in liposomes. In that instance, cleavage of the PEG polymers disrupted the coating of PEG on the surface of the liposomes, resulting in destabilization and rupture of the liposomes, thus releasing their contents.

Cleavage of the hydrophilic polymer or lipid from the conjugates described herein can also be used to alter the HLB of the conjugates in vivo. For example, a more lipophilic compound is favored initially for oral or CNS delivery and for membrane penetration in general. Cleavage of the lipid moiety after the barrier penetration can be used to increase the hydrophilicity and thus the cytosolic solubility of the compound.

The ligand or linking group Z may also comprise an in vivo cleavable moiety, such as an ester, carbamate, carbonate, or disulfide, effective to release the ligand from the conjugate in vivo. This is particularly useful for drug delivery at a target site.

D. Micellar or Liposomal Compositions

Lipopolymers as described herein can be used in micellar or liposomal formulations useful for parenteral delivery. Furthermore, they are potentially useful for blood-brain barrier permeability, as well as for oral delivery.

Liposomes are closed lipid vesicles used for a variety of therapeutic purposes, and in particular, for carrying therapeutic agents to a target region or cell by systemic administration. In particular, liposomes having a surface coating of hydrophilic polymer chains, such as polyethylene glycol (PEG), are desirable as drug carriers, since these liposomes offer an extended blood circulation lifetime over liposomes lacking the polymer coating. The polymer acts as a barrier- to blood proteins, preventing binding of the protein and recognition of the liposomes for uptake and removal by macrophages and other cells of the reticuloendothelial system.

Methods for forming liposomes from lipid components are well known in the art. Liposomes incorporating the lipopolymers of the invention can be prepared by including in a mixture of lipid bilayer components (e.g. phospholipids and/or other vesicle forming lipids) about 1 to about 50 mole percent, preferably about 1 to about 20 mole percent, of the lipid-polymer conjugate of formula I above, where Z is a targeting or therapeutic moiety. In another embodiment, Z is a labeling moiety. Preferably, the lipid-polymer conjugate is of formula II above, i.e. where the lipid is a diacyl glyceryl phospholipid.

The liposomes may contain an encapsulated compound. In one embodiment, the lipopolymer conjugate I or II includes a linkage to the hydrophilic polymer (A) which is cleavable in vivo, such that the hydrophilic polymer is released from the lipopolymer conjugate, preferably after reaching a target site. As described in Kirpotin et al., 1996, the lipid composition of such liposomes can be designed such that the liposomes will be destabilized by loss of the hydrophilic polymer, and will thus release their contents upon in vivo cleavage of the polymer.

### EXAMPLES

The following examples further illustrate the invention described herein and are in no way intended to limit the scope of the invention.

### Example 1

### Synthesis of phosphoramidate-linked Conjugate of 7-hydroxycoumarin and mPEG-DSPE (FIG. 1)

A. Synthesis of mPEG-DSPE-N-1-Boc 13-diaminopropane:

mPEG-DSPE (0.5 g, 0.189 mmol) was dissolved in dichloromethane (5 ml) and cooled to 0° C. under a nitrogen atmosphere. To this solution was added dry DMF (50 μL, 0.646 mmol) and oxalyl chloride (15 μL, 1.72 mmol), and the reaction mixture was stirred at room temperature for 24 hrs. The solvent was evaporated under reduced pressure, and the product was dissolved in dichloromethane (10 ml) and cooled to 0° C. under a nitrogen atmosphere. Triethylamine (200 μL, 1.442 mmol) and N-1-Boc 1,3-diaminopropane (76.45 mg, 0.3628 mmol) were added to the above solution at 0° C. The reaction was stirred in an ice bath for 15 minutes and at room temperature for 15 minutes. TLC (CHCl₃: MeOH: H₂O, 90: 18: 2) showed that reaction had gone to completion (Rf of mPEG-DSPE was 0.533 and of product 0.733). The solvent was evaporated under reduced pressure, and the crude mixture was chromatographed using CHCl₃: MeOH (95:5) as eluent. The product obtained was further precipitated using isopropanol, filtered, and dried over phosphorus pentoxide to give 0.422 g (76%) of the product. ³¹P-NMR (11.01 ppm); ¹H-NMR(DMSO-d⁶) 0.85(t, 6H), 1.23(bs, 56H), 1.36(s, 9H), 1.49(bm, 4H), 2.26(2t, 4H), 2.72(m, 2H), 2.92(q, 2H), 3.23(s, 3H), 3.42(m), 3.50(bs, 180H), 3.67(t, 2H), 3.80(q, 2H), 3.97(m, 2H), 4.04(t, 2H), 4.11(m, 1H), 4.27(m, 1H), 4.95(m, 1H), 5.15(m, 1H), 6.71(t, 1H), 7.32(t, 1H), Maldi (Matrix used: 2,5-dihydroxy benzylic acid+TFA (0.1%)+5 methoxy salicylic acid (1 mg/ml): a distribution at m/z 2958.

B. Synthesis of mPEG-DSPE-7-hydroxycoumarin-1,3-diaminopropane:

mPEG-DSPE-N−1-Boc-1,3-diaminopropane (0.1 g, 0.0337 mmol), prepared as described above, was dissolved in cooled 4N HCl in dioxane (3 ml), and the mixture was stirred at room temperature for 50 minutes. The dioxane was then removed by lyophilization. Formation of the deprotected product was confirmed by TLC (Rf of mPEG-DSPE-N-1-Boc 1,3-diaminopropane was 0.733 and of deprotected product 0.533) and by ³¹P-NMR (13.28 ppm). The deprotected product was dissolved in dichloromethane (2 ml) and cooled to 0° C. To this solution was added N-succinimidyl-7-hydroxycoumarinyl acetate (21.99 mg, 0.069 mmol) in a minimum amount of DMF and triethylamine (23.99 μL, 0.172 mmol). The reaction mixture was stirred at room temperature for 3 hrs under a nitrogen atmosphere. TLC (CHCl₃: MeOH: H₂O, 90: 18: 2) showed the presence of starting material along with a nonpolar product spot (Rf 0.733). The solvent was evaporated under reduced pressure, and the crude product was chromatographed using CHCl₃: MeOH (95:5) as an eluent. The desired product was lyophilized using t-butanol, giving a yield of 40%.

³¹P-NMR (10.99 ppm); 1H-NMR(DMSO-d⁶) 0.85(t, 6H), 1.23(bs, 56H), 1.50(bm, 4H), 2.26(2t, 4H), 2.74(m, 2H), 3.07(m, 2H), 3.20(m, 2H), 3.25(s, 3H), 3.42(m), 3.5(bs, 180H), 3.68(t, 2H), 3.82(q, 2H), 3.67-4.29(m, 6H), 4.98-5.15(m, 1H), 6.15(s, 1H), 6.70(d, 1H), 6.78(dd, 1H), 7.31(t, 1H), 7.58(t, 1H), 7.7(m, 1H), 8.14(t, 1H); Maldi (Matrix used: 2,5-dihydroxy benzylic acid+TFA (0.1%)+5 methoxy salicylic acid (1 mg/ml): a distribution at m/z 3060, epray 1082(3+), 1480(2+). The ¹H and COSY spectra were consistent with the proposed structure shown in FIG. 1.

### Example 2

### Synthesis of phosphoramidate-linked mPEG-protein-phospholipid Conjugate (FIG. 2)

A solution of mPEG-DSPE in dichloromethane was treated with oxalyl chloride and a catalytic amount of DMF. The solution was concentrated by evaporation and the residue redissolved in dichloromethane, cooled on ice, and treated with triethylamine and β-alanine tert-butyl ester. After 15 min the reaction was complete. The solution was concentrated by evaporation, and the β-Ala-O^{t}Bu phosphoramidate product was purified by silica gel chromatography (chloroform-methanol 95:5).

Removal of the tert-butyl ester group was effected by treatment with 4M HCl in dioxane for 6 hrs. The solvent and HCl were removed under vacuum, and the resulting carboxyl-bearing phosphoramidate of mPEG-DSPE was then dissolved in dichloromethane and converted into its succinimidyl ester by reaction with NHS(N-hydroxysuccinimide) and DCC (dicyclohexylcarbodiimide). The formed dicyclohexylurea was filtered and the solution concentrated. The product, mPEG-(β-Ala-OSu)DSPam, was precipitated in isopropanol and characterized by NMR and MS. Overall yield from mPEG-DSPE was approximately 50%.

Lysozyme (2 mg/ml) in phosphate buffer (pH 7.5) was reacted with a 5-fold molar excess of the mPEG-(β-Ala-OSu)DSPam for 1 h. The modified protein was purified by RP-HPLC and characterized by SDS-PAGE and MALDI, confirming the presence of a 1:1 and a 1:2 conjugate of lysozyme and the lipopolymer. The 1:1 conjugate was isolated by cation-exchange chromatography.

Although the invention has been described with respect to particular embodiments, it will be apparent to those skilled in the art that various changes and modifications can be made without departing from the invention.

It is claimed:

1. A compound of the formula: 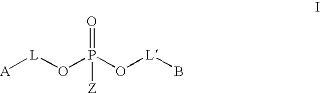
where
A is a hydrophilic polymer;
each of L and L′ is independently a linker group;
B is a lipid moiety; and
Z is selected from the group consisting of a diagnostic ligand, a biologically relevant ligand, and a reactive linking moiety, wherein said reactive linking moiety is not hydroxy (—OH), oxide (—O⁻), or 2-aminoethoxy.

2. The compound of claim 1, wherein B is selected from a fatty acid, a sterol, a diether lipid, and a diacyl lipid.

3. The compound of claim 2, where B is a diacyl lipid, said compound having the formula: 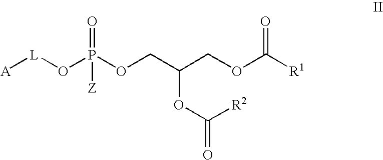
where each of R¹ and R² is independently alkyl or alkenyl having 4-24 carbon atoms.

4. The compound of claim 3, wherein each of R¹ and R² is C₁₇H₃₅.

5. The compound of claim 1, wherein Z is linked to P via a nitrogen, oxygen or sulfur atom in Z.

6. The compound of claim 1, wherein Z is linked to P via a nitrogen atom in Z.

7. The compound of claim 6, wherein Z is a reactive linking moiety of the form —NH—(CH₂)ₙ—X, where n is 2 to 8 and X is selected from amino, mercapto, hydroxy, disulfide, aldehyde, ketone, maleimide, hydrazide, an activated ester, other carboxylic acid derivative, and a leaving group.

8. The compound of claim 6, where n is 3 and X is —NH₂.

9. The compound of claim 6, where n is 3 and X is a succinimidyl ester.

10. The compound of claim 1, wherein Z is a diagnostic ligand.

11. The compound of claim 1, wherein Z is a biologically relevant ligand selected from a polypeptide, a protein, a polynucleotide, and a small molecule compound.

12. The compound of claim 1, wherein A is a polyethylene glycol having 2 to 120 repeating ethylene glycol units.

13. The compound of claim 1, wherein each of L and L′ is independently an alkyl, aryl, or aralkyl moiety, which may be flanked on one or both sides by a group Y, where Y is (i)-W—(C═O)-Q-, (ii)-W—(C═O)—, (iii)-W—, and (iv) disulfide, where W and Q are independently selected from oxygen, NH, and a direct bond.

14. The compound of claim 1, wherein at least one of L and L′ is cleavable in vivo.

15. A liposome comprising a compound according to claim 1.

16. The liposome of claim 15, comprising a compound according to claim 3.

17. The liposome of claim 15, comprising from 1 to about 50 mole percent of the compound according to claim 1.

18. A method of tailoring the hydrophilic-lipophilic balance of a carrier for a drug, comprising
providing a carrier of the formula 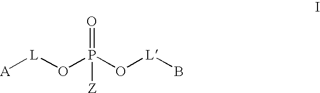
where
A is a hydrophilic polymer,
each of L and L′ is independently a linker group;
B is a lipid moiety; and
Z is said drug or a reactive moiety effective to be conjugated to said drug, wherein said reactive moiety is not hydroxy (—OH), oxide (—O⁻), or 2-aminoethoxy;
and wherein the relative size of A and B is effective to give a desired HLB for said carrier.

19. The method of claim 18, where B is a diacyl lipid, said compound having the formula: 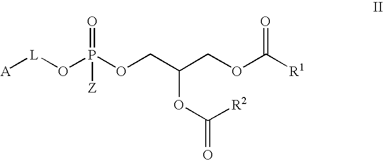
where each of R¹ and R² is independently alkyl or alkenyl having 4-24 carbon atoms.

20. The method of claim 19, wherein A is a polyethylene glycol.

21. A method for oral delivery of a therapeutic agent, comprising
administering orally to a subject a conjugate of the formula I: 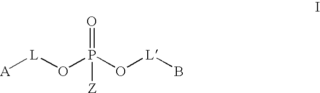
where
A is a hydrophilic polymer;
each of L and L′ is independently a linker group;
B is a lipid moiety; and
Z comprises said therapeutic agent.

22. The method of claim 21, wherein Z further comprises a linkage to the phosphorus atom of formula I which is cleavable in vivo.

23. The method of claim 21, wherein B is a diacyl lipid, such that the conjugate has the formula II: 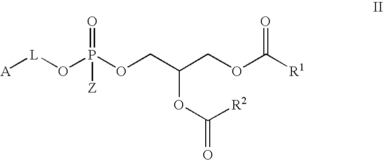
where each of R¹ and R² is independently alkyl or alkenyl having 4-24 carbon atoms.

24. The method of claim 23, wherein A is a polyethylene glycol.
